SEQUENCE LISTING (1 sequence)

SEQ ID NO: 1 - protein, 40 aa, Paraconiothyrium sp.
  misc_feature (6)..(6): Xaa can be any naturally occurring amino acid
AVIDKXLTDDGVPVDVKGSAWGNEDVAPFNIRLPYLXTAI

(19) United States
(12) Patent Application Publication
(10) Pub. No.: US 2010/0056418 A1
(43) Pub. Date: Mar. 4, 2010

(54) DETERGENT COMPOSITION COMPRISING CARBOHYDRATE:ACCEPTOR OXIDOREDUCTASE

(76) Inventor: Neil Joseph Lant, Newcastle (GB)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/548,486

(22) Filed: Aug. 27, 2009

Related U.S. Application Data
(60) Provisional application No. 61/190,305, filed on Aug. 27, 2008.

(30) Foreign Application Priority Data
Mar. 20, 2009 (EP) 09 155 707.4

(51) Int. Cl. C11D 3/386; C12N 9/02

(52) U.S. Cl. 510/320; 435/189

(57) ABSTRACT

The present invention relates to a detergent composition comprising carbohydrate:acceptor oxidoreductase.

### CROSS REFERENCE TO RELATED APPLICATION(S)

This application claims the benefit of U.S. Provisional Application No. 61/190305, filed 27 Aug. 2008.

### FIELD OF THE INVENTION

The present invention relates to detergent compositions comprising carbohydrate:acceptor oxidoreductase. The compositions of the present invention exhibit good bleaching performance. Preferably, the compositions are laundry detergent compositions, most preferably liquid laundry detergent compositions.

### BACKGROUND OF THE INVENTION

The incorporation of bleach into a detergent composition requires a carefully balance of having good bleaching performance and good bleach stability, especially good product storage stability. Whilst many different types of bleach have been considered for incorporation into detergent compositions, such as pre-formed peracids, transition metal bleach catalysts, nitrile quaternary amine bleach activators, cationic imine bleach boosting compounds and the like, there remains very few bleach technologies that exhibit sufficient bleach stability, especially good product storage stability, whilst at the same time providing good bleaching performance and acceptable fabric integrity profile. This is especially true for liquid laundry detergent compositions. Even after decades of development, the commercial yardstick for bleach profile in a solid laundry detergent composition remains, with very few exceptions, percarbonate and/or perborate sources of hydrogen peroxide in combination with bleach activators such as tetraacetylethylenediamine (TAED) and/or alkyloxybenezenesulphonate (AOBS).

With the recent trend in detergent formulation towards increased reliance on improved weight-efficient and cost-effective technologies, developments of bleach catalysis has resurfaced and bleach catalyst technologies such as transition metal catalysts and cationic imine bleach boosting compounds have rekindled. However, these bleach catalyst technologies are used in relatively very small amounts, due to their high weight efficiency.

Using bleach catalysts at these very low levels means that their activity is easily depleted by other components of the detergent composition, such as polyamines. Further instability can arise if the catalysts prematurely activate during storage. This is especially true when the detergent composition is a laundry detergent composition due to the complexity and numerous other chemical components present therein. Also, the bleach stability of the bleach catalyst, and the need to avoid any bleach depletion, is even more critical when the detergent composition is in liquid form, such as a liquid laundry detergent composition.

In addition, using bleach catalysts at very low levels means that it is important that the catalysts are active in the wash when and where bleaching performance is needed. This typically means at the surface to be cleaned. If the bleach catalyst does not reach the surface to be cleaned in any appreciable amount but instead remains in the wash liquor and catalyses bleaching performance in the wash solution, this solution bleaching performance is usually not very efficient as the majority of the available oxygen will be used to bleach soils that are already suspended in the wash liquor and only a minority of the available oxygen will be used to bleach soils that are present at the surface and contribute to the cleaning performance of the detergent composition. This is especially true when the composition is a laundry detergent composition and the surface to be cleaned is a fabric surface.

The Inventor has overcome the above problems and has found a bleach catalyst enzyme that has good stability profile, provides excellent bleaching performance, and exhibits very good bleach efficiency. The inventor has found that a specific carbohydrate:acceptor oxidoreductase generates available oxygen at the fabric surface during a laundering process: the substrate for this specific bleaching enzyme are oligosaccharides arising from cotton as well as other reducing sugars, such as glucose and lactose, present in the fabric stains.

The Inventor has also found that this enzyme provides excellent whiteness benefits due to its effect on the cotton fabric surface which makes the cotton more resistant to soil re-deposition. Without wishing to be bound by theory, the Inventor believes the enzymes acts so as to make the cotton surface more negatively charged (e.g. the enzyme reacts with the terminal glucose moieties of amorphous cellulose and other oligosaccharides of cotton) by oxidizing the terminal reducing ends to a gluconic acid form. This increases the negative charge of the cotton, leading to improved soil repulsion and improved soil anti-redeposition properties of the modified cotton.

### SUMMARY OF THE INVENTION

The present invention provides a composition according to the claims.

### DETAILED DESCRIPTION OF THE INVENTION

### Detergent Composition

The detergent composition can be any detergent composition, such as a laundry detergent composition, dish-washing detergent composition, hard-surface cleaning detergent composition, and the like. Preferably, the composition is a laundry detergent composition.

The composition can be in any form, such as a solid or a liquid. Solid forms typically include granular, flake, noodle, needle and the like. Alternatively the composition can in be the form of a gel, paste, suspension or the like. Preferably, the composition is in the form of a liquid. Most preferably, the composition is a liquid laundry detergent composition.

The composition typically comprises detergent adjunct ingredients.

### Carbohydrate:Acceptor Oxidoreductase

The composition comprises carbohydrate:acceptor oxidoreductase. Preferably, the composition comprises from 0.00015 wt % to 0.50 wt % carbohydrate:acceptor oxidoreductase.

Preferably, the carbohydrate:acceptor oxidoreductase is a parent or variant of the carbohydrate:acceptor oxidoreductase derived from the organism Paraconiothyrium sp. A suitable carbohydrate:acceptor oxidoreductase and its purification method is disclosed in T. Kiryu et al, Biosci. Biotechnol. Biochem., 72(3), 833-841, 2008. The N-terminal partial amino acid sequence of this carbohydrate:acceptor oxidoreductase is given in sequence I.D. No. 1.

The carbohydrate:acceptor oxidoreductase comprises an amino acid sequence that has at least 60 wt %, preferably at least 65 wt %, or at least 70 wt %, or at least 75 wt %, or at least 80 wt %, or at least 85 wt %, or at least 90 wt %, or at least 95 wt % identity to sequence I.D. 1. Preferably the carbohydrate:acceptor oxidoreductase comprises the partial amino acid sequence of sequence I.D. 1.

The amino acid sequence identity is the relatedness between two amino acid sequences described by the parameter “identity”. For purposes of the present invention, the degree of identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends in Genetics 16: 276-277; http://emboss.org), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

### Examples

### Example 1

A liquid laundry detergent composition comprises: 0.05 wt % carbohydrate:acceptor oxidoreductase*, 15 wt % detersive surfactant, 3 wt % fatty acid, 0.3 wt % chelant, 0.2 wt % brightener, 5 wt % solvent, 0.5 wt % perfume, buffer to pH 8.2, water and miscellaneous to 100 wt %.

### Example 2

A solid laundry detergent composition comprises: 0.05 wt % carbohydrate:acceptor oxidoreductase*, 10 wt % detersive surfactant, 10 wt % sodium carbonate, 0.3 wt % chelant, 0.2 wt % brightener, 0.5 wt % perfume, moisture, sodium sulphate and miscellaneous to 100 wt %. *The carbohydrate:acceptor oxidoreductase is the wildtype from the organism Paraconiothyrium sp. disclosed in T. Kiryu et al, Biosci. Biotechnol. Biochem., 72(3), 833-841, 2008.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A detergent composition comprising carbohydrate:acceptor oxidoreductase comprising an amino acid sequence that has at least 60% identity to sequence I.D. 1.

2. A detergent composition according to claim 1, wherein the carbohydrate:acceptor oxidoreductase comprises an amino acid sequence that has at least about 80% identity to sequence I.D. 1.

3. A detergent composition according to claim 1, wherein the carbohydrate:acceptor oxidoreductase is a parent or variant of the carbohydrate:acceptor oxidoreductase derived from the organism Paraconiothyrium sp.

4. A composition according to claim 1, wherein the composition is a laundry detergent composition.

5. A composition according to claim 1, wherein the composition is in liquid form.

6. A composition according to claim 1, wherein the composition is in solid form.

7. A composition according to claim 1, wherein the composition comprises a detersive surfactant.
